(19) United States
(12) Patent Application Publication
(10) Pub. No.: US 2015/0157496 A1
(43) Pub. Date: Jun. 11, 2015

(54) METHOD AND APPARATUS FOR TREATING A SKIN TISSUE

(71) Applicant: KONKLIJKE PHILIPS N.V., EINDHOVEN (NL)

(72) Inventors: Margaret Ruth Horton, Eindhoven (NL); Jonathan Alambra Palero, Eindhoven (NL); Martin Jurna, Eindhoven (NL)

(21) Appl. No.: 14/413,022

(22) PCT Filed: Jun. 24, 2013
(86) PCT No.: PCT/IB2013/055169

§ 371 Date: Jan. 6, 2015

Related U.S. Application Data
(60) Provisional application No. 61/669,174, filed on Jul. 9, 2012.

(51) Int. Cl. A61F 7/02; A61F 5/37; A61F 7/00; A61N 5/00; A61B 18/02

(52) U.S. Cl. CPC A61F 7/02; A61N 5/00; A61B 18/02; A61F 7/007; A61F 5/37; A61F 2007/0075; A61F 2007/0052

(57) ABSTRACT

An apparatus for treating a skin tissue area (1) is provided, comprising: an energy source configured to heat the skin tissue area to a temperature in a range of about 55-65° C., a cooler configured to cool the skin tissue area to a temperature below about 40° C. Further, a skin tissue deformer configured to mechanically deform the skin tissue area into a deformed shape and to maintain the skin tissue area in the deformed shape, and a controller configured to operate the apparatus to perform a method of treating a skin tissue area (1). The method comprises the steps of: mechanically deforming the skin tissue area into a deformed shape; heating the skin tissue area to a temperature in a range between about 55° C. and about 65° C.; cooling the skin tissue area to a temperature below about 40° C. while maintaining the skin tissue area in the deformed shape, and the steps of reheating the skin tissue area to a temperature in a range between about 55° C. and about 65° C. while maintaining the skin tissue area in the deformed shape, and again cooling the skin tissue area to a temperature below about 40° C. while maintaining the skin tissue area in the deformed shape.

### FIELD OF THE INVENTION

The present disclosure relates to treatment of skin, in particular human skin and subdermal tissue. The treatment is primarily suitable for skin tightening and/or skin rejuvenation.

### BACKGROUND OF THE INVENTION

Skin relief features such as wrinkles, especially facial wrinkles, are a sign of aging and generally regarded as unattractive. Wrinkles are physically attributed to folds in the skin that form due to several causes, including photoaging, muscle movement, dehydration, collagen degradation and loss of elastin. Wrinkles have different degrees of severity, ranging from fine lines to deep skin folds. The deeper the skin fold, the more challenging it is to correct. Skin wrinkles contain several features that are apparent with microscopy: thinned epidermis, weakened dermal collagen, amorphous dermal collagen, reduced collagen content and a folded structure.

Topical cosmetic active ingredients can support the synthesis of new collagen, suppress destructive enzymes, support the thickening of the dermis and epidermis, and the repair of the skin's barrier function. Topical cosmetic formulas, depending on the active ingredients, have appreciable efficacy in the treatment of fine lines. However, topical cosmetic formulas are generally unable to correct deep skin folds with high perceived efficacy.

The standard methods of correcting individual deeper wrinkles are to fill up the wrinkles by inserting filling material below the fold so that the fold is flattened, or to modulate the muscular movement by either activating muscles that pull the tissue opposite to the folds for a smoothing effect or immobilizing the muscles that move the skin to form the fold. However, these methods require transcutaneous needles and are therefore invasive and undesirable to many patients. Additionally, neuromodulators can diminish the capacity to create facial expressions, which can be undesirable.

Alternative methods of correcting wrinkles do not target individual wrinkles, but rather target the full face and include chemical peeling, dermabrasion, laser skin resurfacing and fractional laser skin resurfacing. The general mechanism is that the skin of the entire treatment area is injured, either with full area coverage or in a fine fractional pattern, stimulating the formation of new collagen. Here the damage to the tissue is irreversible and new tissue is generated. The resulting rejuvenated skin is denser and firmer and the appearance of wrinkles is diminished as the wrinkles' tissue is replaced with new tissue. In these methods, the tissue injury usually targets the dermis, which comprises a dense collagen network that is responsible for giving the skin its shape.

A promising approach to address undesired skin features is presented in US 2008/0306476, which discloses a method and a device for modifying tissue in such a manner that it will take on a permanent new shape. Subdermal tissue is heated percutaneously to approximately 60° C., such that it becomes pliable and it will take on a permanent new shape if allowed to cool and heal in a new position. The heated dermal tissue is molded to a new desired shape. The dermis is then held in the desired new shape until it cools and retains the new shape. The heating of the dermal tissue is performed using a percutaneous probe.

However, it has been found that the tissue is not completely shaped by the short term collagen cross-linking, and a major increase of the strength and further deformation take place as new collagen is formed in the molded tissue and other affected tissue over 12 months post treatment. The further deformation may comprise marks of tight clothes.

Methods and apparatus for modifying skin tissue providing a more permanent and reliable result are therefore desired.

### SUMMARY OF THE INVENTION

In an aspect, a method of treating a skin tissue area according to the appended claims is provided.

The method comprises the steps of mechanically deforming the skin tissue area into a deformed shape, heating the skin tissue area to a temperature in a range between about 55° C. and about 65° C., cooling the skin tissue area to a temperature below about 40° C., preferably to or near normal body temperature, while maintaining the skin tissue area in the deformed shape, reheating the skin tissue area to a temperature in a range between about 55° C. and about 65° C. while maintaining the skin tissue area in the deformed shape, and cooling the skin tissue area to a temperature below about 40° C., preferably to or near normal body temperature, while maintaining the skin tissue area in the deformed shape.

In the temperature range of about 55-65° C., reversible denaturation of human collagen occurs. At temperatures below about 50° C., the collagen is substantially unaffected by heating. At temperatures in the range of about 55-60° C. denaturation within and between collagen fibrils starts to occur, and at temperatures between about 60° C. and 65° C. substantially all collagen is denatured while remaining viable, and tissue comprising the collagen becomes deformable. The texture of skin tissue in such a situation is likened to warm clay. Once deformed and cooled down to normal body temperature, the tissue generally retains the new shape but is otherwise undamaged and viable. At temperatures above about 65° C., the denaturation tends to become irreversible with the collagen losing its elasticity and collapsing, leading to contraction. Such temperatures and their effects are avoided in the present method, in which human skin tissue should not be heated to a temperature higher than about 65° C. The high temperature may be maintained for some time, e.g. several seconds to minutes, to facilitate a more complete collagen denaturation, possibly denaturation of substantially all collagen in the tissue area.

Cooling the skin tissue area to a temperature below about 40° C., in particular close to or below normal body temperature, in any case significantly below the temperature of the onset of collagen denaturation, e.g. to a temperature in a range of about 40-25° C., enables (re)creation of links within and between denatured collagen fibrils so that the tissue stabilizes into a new permanent shape. Cooling to normal body temperature (typically 30-35° C. for skin tissue) or somewhat below after the initial heating step is preferred for (re)establishing collagen links associated with the deformed shape. By maintaining the skin tissue area in the deformed shape during the cooling of the tissue, the deformed shape becomes more accurately “frozen into the tissue” and relaxation to the original shape is prevented. Although natural cooling, e.g. by physiological processes and/or natural convection, occurs when the application of heat to the skin tissue area is stopped, forced cooling of the tissue is considered important for reliable determination and fixation of the final shape of the cooled tissue.

Reheating and again cooling the tissue portion while maintaining the tissue in the deformed shape promotes disruption of unfavourable, remaining or newly-formed, collagen links and results in the formation of more and/or stronger collagen links associated with the new shape. Thus, a significantly better fixation of the deformed shape into the tissue is achieved as compared to a single cycle. This is comparable, to some extent, to an annealing procedure to reduce internal stress in solids. As a result, the treated tissue regains a firm and permanent structure relatively fast and undesired (further) deformation of the tissue such as a (partial) return to the initial shape and/or deformation under subsequent forces such as tight clothes is reduced or even prevented.

During the heating and/or reheating step(s), the high temperature may be maintained for some time, e.g. several seconds to minutes, to facilitate a more complete denaturation of the collagen, possibly denaturation of substantially all collagen in the tissue area. Similarly, when, during the cooling and/or re-cooling step, the skin tissue is cooled to a temperature below normal body temperature, such a low temperature may be maintained for some time, e.g. several seconds to minutes, to increase the number and/or strength of collagen links within and between collagen fibrils in the tissue area.

Collagen tends to contract when maintained at temperatures between about 55° C. and about 60° C., as a result of which shrinkage by 10% or more is generally possible. Such shrinkage may have a permanent nature. Without external forces to deform (skin tissue comprising) the collagen and/or maintain a deformed shape, such shrinkage may counteract desired and intended deformation. Heating the tissue to a temperature higher than such a contraction temperature, such as in the range of about 60-65° C., facilitates that the intended “melting” effect of the collagen dominates possible contraction, and cooling the tissue from the high temperature to a temperature well below 55° C. is therefore employed. Similarly, rapid heating and cooling to reduce the time that the tissue is at a temperature in the range between about 55° C. and 60° C. where contraction may dominate the “melting” is preferred.

With the presently provided method, non-invasive reshaping of skin features is achieved that can provide a younger appearance. Since the treated skin tissue remains viable, the method can be repeated without adverse effects and correction and/or further deformation of a previously treated skin tissue area is possible if so desired.

For further reliability of the method and improved fixation of the deformed shape, the method may comprise repeating, one or more times, the steps of reheating the skin tissue area to a temperature in a range between 55° C. and 65° C. while maintaining the skin tissue area in the deformed shape, and again cooling the skin tissue area to a temperature below about 40° C. while maintaining the skin tissue area in the deformed shape.

The step of mechanically deforming the skin tissue area into a deformed shape is advantageously performed during and/or before the step of heating the skin tissue area. Mechanically deforming the skin tissue area during the heating step allows taking advantage of the malleability of sufficiently heated tissue to provide a desired deformed shape. Mechanically deforming the skin tissue area before the heating step and maintaining the skin tissue area in the deformed shape during the heating step is preferred because this facilitates disrupting unfavourable collagen links already at the first heating step and thus increases the effectiveness of the method.

The method may comprise application of radiofrequency energy to the skin tissue area for heating and/or reheating the portion of skin tissue. The use of radiofrequency energy for heating skin is a proven and trusted technique which can be accurately controlled so that negative side effects can be prevented. The radiofrequency energy may be applied non-invasively, which may increase acceptance of the method and/or prevent (risks of) complications from healing processes. However, other techniques such as heating by electrical resistance, ultrasound and/or electromagnetic waves in the optical and/or microwave range may be suitably employed. It is noted that the heating energy need not be applied uniformly and/or over the entire skin tissue area to be treated, because a dispersed fractional pattern, possibly having a temporal variation, is also possible.

In a typical embodiment, the skin tissue area comprises a skin tissue relief feature, such as a wrinkle or a scar, and the step of mechanically deforming the skin tissue area into a deformed shape comprises stretching the skin tissue area comprising the skin tissue relief feature. Thus, the amplitude of the skin relief feature, e.g. the depth of a wrinkle, may be reduced and a smoother and more youthful appearance may be provided.

The step of mechanically deforming the skin tissue area into a deformed shape may comprise deforming at least a portion of the skin tissue area, using a mechanical skin stretcher. In an embodiment, the skin tissue area may be maintained in the deformed shape when using a mechanical skin stretcher. Use of a mechanical skin stretcher facilitates flattening of skin and providing and/or maintaining a desired deformed shape in a reliable manner, also in the case of prolonged and/or repetitive application (of treatment cycles) of the method.

The method may further comprise at least one of the steps of dermabrasion, micro dermabrasion, application of microlesions and outer skin layer resurfacing to promote rejuvenation of the skin tissue.

In accordance with the above, in an aspect an apparatus for treating a skin tissue area is provided, comprising an energy source configured to heat the skin tissue area to a temperature in a range of about 55-65° C., a cooler configured to cool the skin tissue area to a temperature below about 40° C., a skin tissue deformer configured to mechanically deform the skin tissue area into a deformed shape and maintain the skin tissue area in the deformed shape, and a controller configured and arranged to operate the apparatus so as to mechanically deform the skin tissue area into a deformed shape by application of the skin tissue deformer, heat the skin tissue area to a temperature in a range of about 55-65° C. by application of the energy source, subsequently cool the heated skin tissue area to a temperature below about 40° C. by application of the cooler while maintaining the skin tissue area in the deformed shape by application of the skin tissue deformer, subsequently reheat the skin tissue area to a temperature in a range of about 55-65° C. by the energy source while maintaining the skin tissue area in the deformed shape by application of the skin tissue deformer, and subsequently cool the skin tissue area to a temperature below about 40° C. by application of the cooler while maintaining the skin tissue area in the deformed shape by application of the skin tissue deformer. With the apparatus, the method as described herein may be suitably performed and skin tissue can be permanently deformed in vivo without adverse consequences. The deformer may in particular be configured to stretch the skin tissue area, e.g. to reduce or remove one or more wrinkles and flatten the skin tissue area.

The apparatus comprises a cooler configured to cool the skin tissue area to a temperature below about 40° C., preferably to or near normal body temperature or below. Thus, forced cooling, at least faster than by physiological processes of the body, can be provided to the skin tissue to increase treatment efficiency, reduce treatment duration and/or facilitate prevention of undesired contraction of the collagen.

The skin tissue deformer may comprise a plurality of probes configured to engage a surface of the skin tissue area, the probes being movable with respect to each other to thereby form the skin tissue area into a deformed shape. The probes may be elements providing a high coefficient of friction when in contact with human skin surface, e.g. comprising a rubber and/or roughened contact surface for contacting human skin, and/or one or more vacuum cups configured to engage the skin by suction. The skin can also be compressed with positive pressure to achieve the desired deformation. Thus, skin tissue may be pushed and/or pulled in a desired direction to flatten the skin tissue area. Such a deformer facilitates maintaining a particular deformation for extended periods of time in a non-invasive manner. The deformer may be adjustable to establish and/or maintain a particular deformed shape.

In an embodiment, a plurality of probes are connected, preferably resiliently, with respect to each other, wherein the probes have contact surfaces for contacting the surface of the skin tissue area, and the probes being arranged to separate from one another upon being pressed against the skin surface.

The cooler may comprise a heat sink, such as a radiator with a high thermal conductance and a large surface, and/or a cryogenic cooling element, but preferably the cooler is an active cooling element such as a Peltier-element and/or a refrigerator device, which may be controllably operated.

The controller may be configured to operate the apparatus as a function of one or more input signals, e.g. from a user interface. In an embodiment, the controller may be configured to operate the deformer, e.g. to provide or maintain a particular deformed shape.

The apparatus may comprise a thermometer configured to detect a temperature of the skin tissue area and the controller may be configured to operate the apparatus, in particular the energy source, as a function of one or more signals from the thermometer. This facilitates controlled operation by, for example, providing safety against overheating and/or including a feedback mechanism.

The apparatus may comprise a profilometer, e.g. to provide information on a height profile of a skin tissue relief feature and the controller may be configured to operate the apparatus, in particular the skin tissue deformer, as a function of one or more signals from the profilometer. The profilometer may be configured to provide one or more signals such as visual indications and/or signals to be used as input signals to a controller configured to operate the apparatus as a function of one or more signals from the profilometer, e.g. for adjustment of the deformer. The profilometer can also provide information on the progress and/or effectiveness of the method, e.g. via an indication of the apparatus such as via a user interface. For increased accuracy, the profilometer data may be compared to associated data of the skin tissue area prior to deformation and/or of un-deformed skin tissue portions adjacent the deformed skin tissue area. A suitable apparatus for performing the method may comprise a profilometer coupled with a memory for storing reference data and a controller for comparing measurement data with stored reference data. It is conceivable that a plurality of heating, cooling, reheating and re-cooling cycles are performed according to the method presented herein until a particular skin tissue profile is reached, which may be based on data provided by the profilometer. An additional reheating and re-cooling cycle may be performed automatically, based on one or more preset criteria and/or may be decided by a user, e.g. based on an indication of the apparatus suggesting such a further cycle.

The profilometer may comprise a mechanical detector, e.g. with one or more mechanical probes, an electrical detector, e.g. with one or more capacitive or resistive sensors and/or an optical detector, e.g. with an optical reflectance sensor, a camera etc. In addition to surface relief information, the profilometer may measure, image and/or provide feedback on deeper tissue structures within the skin.

The apparatus may be configured for performing dermabrasion, micro dermabrasion, and for the application of microlesions and/or outer skin layer resurfacing to the skin tissue area, for which purpose the apparatus comprises, for example, a mechanical skin tissue perforator, a suitable light source, an ultrasound generator etc. Such apparatus facilitates inducing skin rejuvenation in addition to providing a reformed skin shape to provide a smoother, younger-looking skin.

### BRIEF DESCRIPTION OF THE DRAWINGS

In the drawings:

FIGS. 1-5 show steps of an embodiment of a method of treating a skin tissue;

FIG. 6 shows an exemplary temperature characteristic of the method;

FIGS. 7-8 show an embodiment of an apparatus for treating a skin tissue area.

### DETAILED DESCRIPTION OF EMBODIMENTS

It is noted that in the drawings, like features may be identified with like reference signs. It is further noted that the drawings are schematic, not necessarily to scale and that details that are not required for understanding the present invention may have been omitted. The terms “upward”, “downward”, “below”, “above”, and the like relate to the embodiments as oriented in the drawings. Further, elements that are at least substantially identical or that perform an at least substantially identical function are denoted by the same numeral, possibly raised by 100, 200, etc.

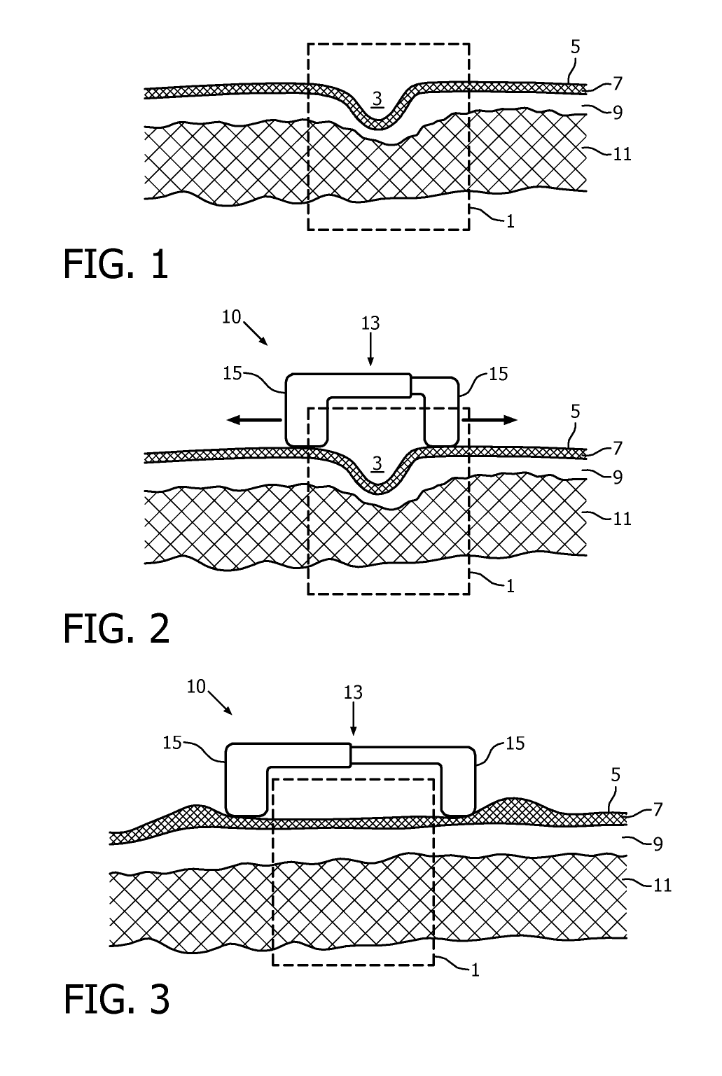

FIGS. 1-5 indicate a method of treating a human skin tissue area 1, here comprising a skin relief feature 3 in the form of a depression such as a wrinkle (FIGS. 1-2). The skin tissue 1, shown in cross section, comprises a stratum corneum 7 providing a skin surface 5, an epidermis layer 9 and a dermis layer 11. Tissue underneath the dermis layer 11 is not shown.

For treating the skin tissue area 1, an apparatus 10 comprising a skin tissue deformer 13 is placed on the skin surface (FIG. 2). The shown skin tissue deformer 13 comprises probes 15, which comprise a contact surface capable of providing high friction to the skin surface because they, e.g, have a rough surface or contain a silicone rubber, etc. The probes 15 are movable with respect to each other from a first separation (FIG. 2) to a second, larger, separation (FIG. 3). Due to the frictional engagement of the probes 15 with the skin surface 5, the skin tissue area is stretched. Thus, the skin tissue area 1 is mechanically deformed from its initial shape (FIGS. 1-2) to a deformed shape (FIGS. 3-5). In the shown embodiment, in the deformed shape the skin relief feature 3 is largely flattened.

The deformer 13 is configured such that the deformed shape of the skin tissue area may be substantially maintained for prolonged periods of time throughout the carrying out of the method.

FIG. 4 shows the use of a thermal energy source 17, e.g. an electrical resistance heater, to heat the skin tissue area 1, such that at least the dermis layer 11 is heated to a temperature in a range, in this embodiment, between about 60° C. and about 65° C., while maintaining the skin tissue area 1 in the deformed shape. Thus, the heated collagen in the skin tissue area 1 is “melted” by the heat and adapts to the deformed shape. Other suitable heaters comprise ultrasound-, radiofrequency-, microwave, and/or other electromagnetic sources; laser light, intense pulsed light, broadband light or other light sources may also be used for heating. The energy source may be configured to heat the skin tissue by providing an energy form selected to be predominantly absorbed by the collagen, e.g. using a particular optical wavelength range.

The heating energy can be applied uniformly over the skin treatment area, or dispersed in a fractional pattern over the skin treatment area. Said heating energy can be applied using short pulses (e.g. <1 milliseconds) or a sustained long pulse (e.g. 1-5 seconds).

It is noted that a depth profile of the heating may be provided by the application of a heating method providing a large penetration depth for the heat, such as radiofrequency heating and/or heating with infrared light and/or for a prolonged duration, combined with superficial cooling of a more exterior tissue area. Such a combination can cause heating of the deeper skin tissue portions to a high temperature while reducing or even preventing a hot feeling and/or a feeling of pain.

FIG. 5 shows a subsequent step of cooling, by means of a cooler 19, the heated skin tissue area 1, or at least the dermis layer 11, to a temperature of about normal body temperature, but at least below about 40° C., while maintaining the skin tissue area 1 in the deformed shape. Thus, the collagen can regain firmness and the deformed shape is “frozen” into the skin tissue area 1.

Next, the steps of heating and cooling (FIGS. 4 and 5) are repeated one or more times to strengthen the collagen bonds of the skin tissue in the deformed state and imprint the deformed shape into the skin more definitively. After completion of the desired number of repetition cycles and after cooling the skin tissue area 1 to a desired temperature, the deformer 13 may be removed from the skin tissue area 1, which will retain the deformed shape.

Since the final shape of the treated skin tissue area 1 is determined by the heated tissue, predominantly by the portion of the dermis layer 11 in it, any skin relief features formed in the skin tissue outside of the heated zone, such as bulges or wrinkles, by outward action of the deformer 13, will not be affected and imprinted into the skin tissue area 1, so that they are transient and of no concern.

A controller (not shown) is provided in order to control operation of the apparatus 10 to control the amount of deformation, prevent overstretching, overheating and/or undercooling of the skin tissue area. Suitable skin tissue properties that may provide input parameters for controlling the operation of the apparatus may comprise the temperature, flatness, firmness/softness, elasticity of the skin tissue area and/or the colour of the skin surface, etc.

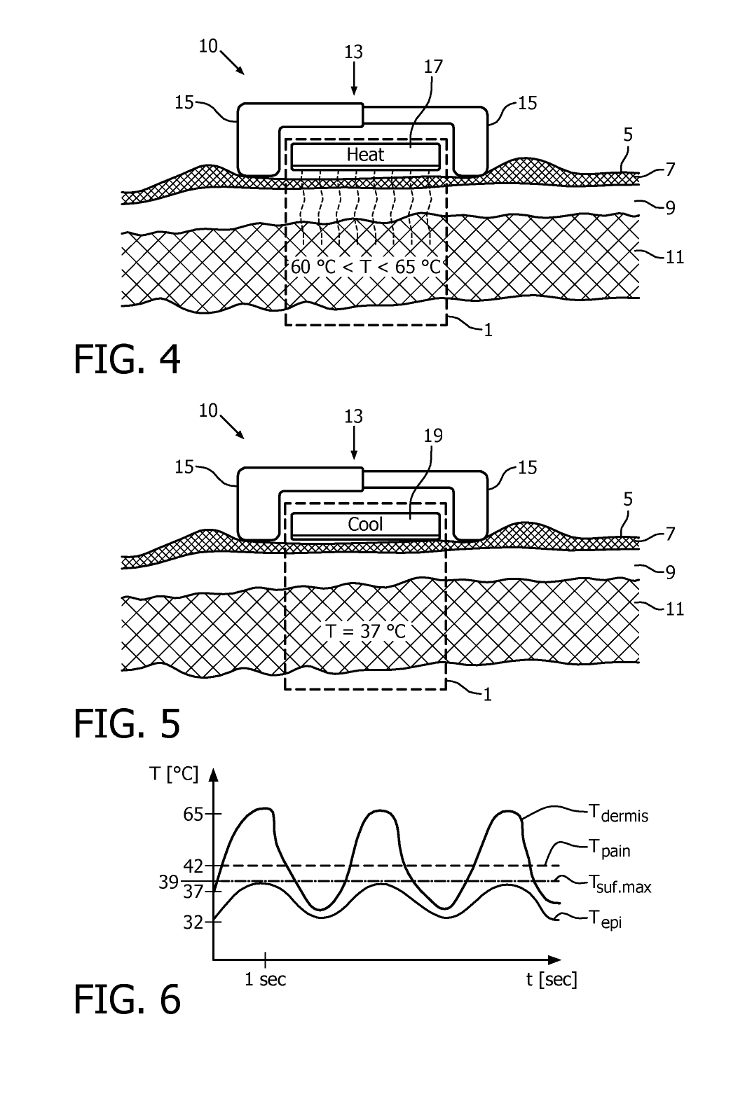

FIG. 6 is a graph showing an exemplary temporal evolution of the skin temperature profile of a treatment with an initial heating and cooling cycle and two, substantially identical, repetitions. The graph shows the dermal temperature T_{dermis}, the skin surface temperature Tₑₚᵢ, the pain threshold on the skin surface Tₚₐᵢₙ and the maximum surface temperature achieved by performing the method T_{surf.max}. From the graph, the following may be appreciated: the dermis temperature T_{dermis} is raised from the normal skin temperature of about 37° C. to a peak temperature of about 65° C. within a period of about 1 second, after which the skin is rapidly cooled in about 1 second, and subsequently the cycle is repeated twice. During the treatment, the epidermis is heated from the normal skin temperature of about 32° C. to a maximum temperature of about 39° C., so that the skin surface temperature remains lower than the pain threshold Tₚₐᵢₙ at about 42-45° C., and hence the treated subject does not feel pain. It has been found that monitoring of the skin surface temperature with a thermometer provides sufficient information on the dermis temperature T_{dermis}, so that the apparatus may be readily provided with a suitable control system to prevent overheating, without having to use invasive measurement techniques. Suitable thermometers may comprise contact thermometers and/or optical thermometers.

It is noted that the heating may be performed at a relatively low rate of increase, e.g. a characteristic rate of increase of the order of seconds, whereas cooling may preferably be performed rapidly, faster than by physiological processes, e.g. at a rate of the order of 0.1 s or faster like 50 ms, so as to stop the heating process abruptly and prevent overheating of the skin tissue and/or to increase the effect of “freezing in” the deformed shape into the collagen bonds.

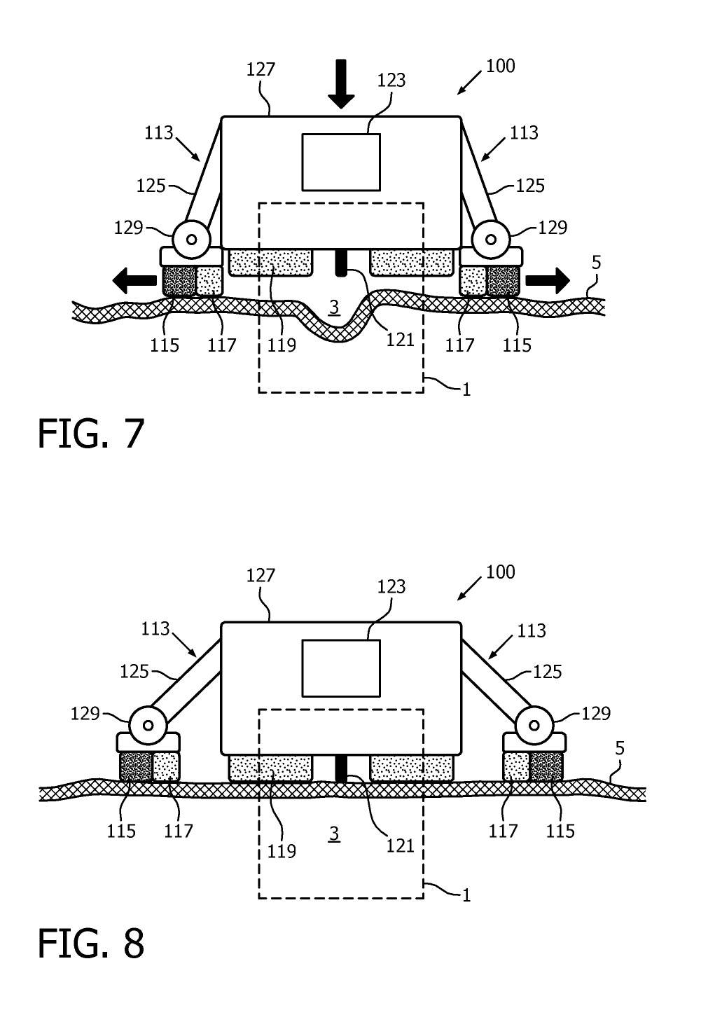

FIGS. 7 and 8 show an embodiment of an apparatus 100 for performing the method, said apparatus being placed on a skin tissue area 1 to be treated having a skin relief feature 3. The apparatus 100 integrates a skin tissue deformer 113, a skin heating energy source 117, and a cooler 119 in the single apparatus 100. The apparatus 100 further comprises a thermometer 121 and a controller 123 for controlling operation. The apparatus 100 may comprise a user interface (not shown) for controlling operation, possibly in combination with the controller 123, and/or an external and/or an internal power source (not shown) for increased mobility of the apparatus 100.

The skin tissue deformer of the apparatus 100 comprises friction probes 115 arranged on arms 125 extending from a main body 127 of the apparatus 100 for contacting the skin surface 5 and stretching the skin tissue area 1 to be treated by moving outward with respect to each other from a first separation (FIG. 7) to a second, larger, separation (FIG. 8). Thus, the skin tissue area 1 is mechanically deformed (here: flattened) from its initial shape (FIG. 7) to a deformed shape (FIG. 8). Suitably, the arms 125 force the probes 125 to move away from each other by merely bringing the probe surfaces into contact with the skin tissue area 1 to be treated and pressing the main body 127 of the apparatus 100 towards the skin surface 5 (cf. bold arrows in FIG. 7), wherein in this embodiment the probes 115 can assume an appropriate orientation with respect to the skin surface 5 by means of optional hinges 129. The second separation may be adjustable by adjustment of the arms 125. However, other deformers may be envisioned, including a deformer that is controllable by a controller. In an embodiment (not shown), the deformer may deform different amounts of skin tissue in different directions, e.g. for deforming irregularly shaped scar tissue to a smooth structure.

In the present embodiment, coolers 119 are arranged at an underside of the main body 127, so that when the main body 127 of the apparatus 100 is pressed towards the skin surface 5, the coolers 119 contact the skin surface 5 (FIG. 8). A thermocouple acting as the thermometer 127 and being coupled to the controller 123 also contacts the skin in this configuration.

Adjacent the probes 115, and on an inside thereof with respect to the opposing probes 115, radiofrequency (RF) electrodes 117 are arranged as energy sources for heating the skin tissue area 1, said radio frequency (RF) electrodes being connected with a suitable RF energy source of the apparatus 100, preferably in the main body 127. RF energy tends to flow predominantly through the dermis layer, and in particular in combination with superficial cooling of the epidermis by the coolers 119, e.g. Peltier elements, the apparatus 100 readily provides the desired thermal profile in the skin tissue area 1. The coolers 119 may be controlled to operate at low power during heating of the skin tissue by the RF energy and at a higher power to cool the skin tissue in the cooling phase of the method. However, heating without superficial cooling is also conceivable.

In a typical apparatus, in the stretched configuration, the electrodes are spaced 0.5-2 cm apart. The RF energy can have a frequency in a range of 0.4-100 MHz, and a power of 1-150 W. RF energy enables volumetric dermal heating for an individual skin feature with the described electrode spacing.

The apparatus may comprise one or more sensors connected with the controller to monitor at least one of skin temperature (see above), skin color, skin blood perfusion, skin resistance or capacitance, acoustic or mechanical vibration characteristics of the skin, and ultrasound reflection of the skin. Ultrasound imaging and/or polarization microscopy may also be used to obtain information on properties of skin and progress of the method.

The apparatus may comprise one or more devices configured for performing dermabrasion, microdermabrasion, and for the application of microlesions and/or outer skin layer resurfacing to the skin tissue area 1.

A deformer may be configured to deform skin in an elongated, e.g. linear, pattern, e.g. for treating elongated wrinkles, and/or in a generally circular pattern, e.g. for treating local skin relief features such as ice-pick acne scars, cellulitis marks, dimples etc. A deformer may comprise a plurality of skin engagement structures for defining more complex shapes of skin tissue areas to be treated.

A larger or smaller number of heaters, coolers, deformers and/or other elements may be provided.

An embodiment may include a sliding or repositioning mechanism in the above-described apparatus in a plane parallel to the skin, allowing one area of the skin feature to be treated, and subsequently an additional area.

In another embodiment, after at least two areas of skin have been treated, the apparatus is relocated to a previously-treated area, which has reached the desired cooling temperature, and an additional heating/cooling cycle is executed.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different embodiments and/or dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. A method of treating a skin tissue area comprising the steps of:
mechanically deforming the skin tissue area into a deformed shape,
after the step of mechanically deforming the skin tissue area into the deformed shape, heating the skin tissue area to a temperature in a range between about 55° C. and about 65° C. while maintaining the skin tissue area in the deformed shape,
cooling the skin tissue area to a temperature below about 40° C. while maintaining the skin tissue area in the deformed shape,
reheating the skin tissue area to a temperature in a range between about 55° C. and about 65° C. while maintaining the skin tissue area in the deformed shape, and
again cooling the skin tissue area to a temperature below about 40° C. while maintaining the skin tissue area in the deformed shape.

2. The method of claim 1, further comprising repeating the step of reheating the skin tissue area to a temperature in a range between about 55° C. and about 65° C. while maintaining the skin tissue area in the deformed shape, and the step of cooling the skin tissue area to a temperature below about 40° C. while maintaining the skin tissue area in the deformed shape.

3. (canceled)

4. (canceled)

5. The method of claim 1, wherein at least one of the steps of heating and reheating the skin tissue area comprises application of radiofrequency energy to the skin tissue area.

6. The method of claim 1, wherein the skin tissue area comprises a skin tissue relief feature and wherein the step of mechanically deforming the skin tissue area into a deformed shape comprises stretching the skin tissue area comprising the skin tissue relief feature.

7. The method of claim 1, wherein the step of mechanically deforming the skin tissue area into a deformed shape comprises deforming at least a portion of the skin tissue area using a mechanical skin stretcher.

8. The method of claim 1, further comprising at least one of the steps of dermabrasion, microdermabrasion, application of microlesions, and outer skin layer resurfacing.

9. An apparatus for treating a skin tissue area comprising:
an energy source configured to heat the skin tissue area to a temperature in a range of about 55-65° C.,
a cooler configured to cool the skin tissue area to a temperature below about 40° C.,
a skin tissue deformer configured to mechanically deform the skin tissue area into a deformed shape and maintain the skin tissue area in the deformed shape, and
a controller configured and arranged to operate the apparatus to perform the method of any one of the preceding claims.

10. The apparatus according to claim 9, wherein the skin tissue deformer comprises a plurality of probes configured to engage a surface of the skin tissue area, the probes being movable with respect to each other to thereby mechanically deform the skin tissue area into a deformed shape.

11. The apparatus according to claim 9, wherein the cooler comprises a radiator, a heat sink, a Peltier-element, a cryogenic cooling element and/or a refrigerator device.

12. The apparatus according to claim 9, comprising a thermometer configured to detect a temperature of the skin tissue area, wherein the controller is configured to operate the apparatus, in particular the energy source as a function of one or more signals from the thermometer.

13. The apparatus according to claim 9, comprising one or more radiofrequency electrodes, the apparatus being configured to heat and/or reheat the skin tissue area by application of radiofrequency energy to the skin tissue.

14. The apparatus according to claim 9, comprising a profilometer, wherein the controller is configured to operate the apparatus, in particular the skin tissue deformer, as a function of one or more signals from the profilometer.

15. The apparatus according to claim 9, being configured for performing dermabrasion, microdermabrasion, and for the application of microlesions and/or outer skin layer resurfacing to the skin tissue area.
